(19) United States
(12) Patent Application Publication
(10) Pub. No.: US 2007/0078417 A1
(43) Pub. Date: Apr. 5, 2007

(54) METHOD AND SYSTEM FOR SYSTEMATICALLY TREATING HEMORRHOIDS

(76) Inventor: J. Stephen Bohan, Vineyard Haven, MA (US)

Correspondence Address: BARLOW, JOSEPHS & HOLMES, LTD., 101 DYER STREET, 5TH FLOOR, PROVIDENCE, RI, 02903, US

(21) Appl. No.: 11/538,545

(22) Filed: Oct. 4, 2006

Related U.S. Application Data
(60) Provisional application No. 60/723,365, filed on Oct. 4, 2005.

(51) Int. Cl. A61M 35/00

(52) U.S. Cl. 604/304; 604/289; 206/440

(57) ABSTRACT

A novel method and system for a systematic approach to the treatment of hemorrhoids is provided. The system is a coherent and targeted treatment regimen for the comprehensive treatment for hemorrhoid sufferers. The system of the present invention includes the following elements: 1) Educational materials about hemorrhoids including the prevention and treatment thereof; 2) Cream for use in early stages of development for relief of minor symptoms in a manner that aids in preventing progression to more uncomfortable stages requiring surgical intervention; 3) Fiber pad wipes; 4) Cleanser; and 5) Optionally, a prescription strength ointment for relief of moderately severe symptoms of pain and swelling, the use of which can prevent progression to a stage requiring surgery.

### CROSS-REFERENCE TO RELATED APPLICATIONS

This application is related to and claims priority from earlier filed US Provisional Patent Application No. 60/723,365, filed Oct. 4, 2005, the contents of which are incorporated herein by reference.

### BACKGROUND OF THE INVENTION

The present invention relates generally to a method for treating hemorrhoids. More specifically, the present invention is directed towards a collection of treatment means that have been formulated into a comprehensive regimen for the systematic treatment and prevention of hemorrhoids.

Hemorrhoids are a common condition that affects approximately 15 million people or about 5% of the adult population. Typical symptoms include itching, bleeding and pain. The symptoms associated with hemorrhoids are the result of the swelling of blood vessels at the site where the two venous systems in the body, the systemic and portal systems, are conjoined. Swelling or dilation of these blood vessels may result from an increased pressure in the hemorrhoidal vein. Constipation, including the frequent straining to pass hard stools, can result in increases pressure in hemorrhoidal veins and is one common cause of hemorrhoids. Other factors that may contribute to increased swelling in the region include pregnancy, a low fiber diet, and obesity. As the hemorrhoidal vein becomes more dilated from the increased pressure, it may eventually form a sac-like protrusion if the condition is allowed to persist. Hemorrhoids are generally classified as being either internal or external, depending on their location relative to the dentate line. The dentate line is easily identified as the demarcation between the pink mucosa that forms the anoderm. The dentate line separates the internal and external hemorrhoid systems. Internal hemorrhoids are located inside the anus above the dentate line. External hemorrhoids are located below the dentate line. Either can extend out of the anus.

Further straining or irritation caused by passing stool can injure the delicate surface of an internal hemorrhoid and cause bleeding. If the pressure and dilation of the hemorrhoidal vein continues, the internal hemorrhoids may prolapse and be forced through the anal opening. If a hemorrhoid remains prolapsed, considerable discomfort, including itching and bleeding, may result. The blood supply to these prolapsed hemorrhoids may become cut off by the anal sphincter, which gives rise to a strangulated hemorrhoid. Thrombosis may result where the blood within the prolapsed vein becomes clotted. This extremely painful condition can cause edema and inflammation.

Often the pain associated with hemorrhoids may be great enough to interfere with normal patterns of defecation which in turn leads to constipation and further aggravation of the swelling in the region. In general most symptoms associated with hemorrhoids are sufficient enough to interfere with a person's daily life causing them to require relief from a medical practitioner and/or a pharmaceutical preparation.

There is a wide range of therapies available that have been used in an incremental fashion in an attempt to provide a person suffering from hemorrhoids with some level of relief. Therapies range from medicated ointments and creams to surgery.

There are in fact many creams and ointments that have previously been developed which generally relieve either the itching or inflammation but few have been successful in reducing or completely eliminating both. The traditionally available creams however contain no product that has been shown to be effective for treatment of hemorrhoids.

Thus, the efficacy of these compositions in relieving or curing the symptoms of such diseases is uncertain

Alternatively, treatment via surgery ranges from minor surgery to relieve symptoms through drainage of the swelling to major invasive surgery. For example, one surgical option includes the removal of extensive or severe hemorrhoids in a process known as a hemorrhoidectomy. This surgical procedure can be used on both internal and external hemorrhoids. However, such surgery typically involves a long recovery period, along with the associated risks and expense of invasive surgery.

Internal hemorrhoids may be treated by rubber band ligation, where a legator is inserted through a scope in the anal canal. The hemorrhoid is grasped with forceps in the legator and held in position. The legator includes a cylinder that is slid upwards and releases one or more rubber bands around the base of the hemorrhoid. A typical diameter for the rubber band is one millimeter. The band cuts off the circulation of blood to the hemorrhoid, and the hemorrhoid begins to wither away. Provided the rubber band remains in place, the hemorrhoid typically drops off within seven to ten days.

Sclerotherapy, another treatment for hemorrhoids, involves injecting a solution, such as sodium morrhuate or phenol oil, submucously into the areolar tissue around the hemorrhoidal vein to cause inflammation and scarring that in turn eliminates the hemorrhoid. Other external treatments cause burning or coagulation to destroy the hemorrhoid. In infrared coagulation, infrared light may be applied to create a small tissue-destroying burn around the base of the hemorrhoid to cut off the blood supply to the hemorrhoid. Electrocoagulation, sometimes referred to as bipolar diathermy, may be utilized in a similar manner. In laser therapy, also known as vaporization, a laser beam causes a superficial burn to seal off the blood vessels and retain the hemorrhoid in a non-prolapsed position.

While a broad range of therapies have been employed, the problem is that there is no real consensus among the medical community as to the best form of therapy. As was stated above, none of the topical applications are actually proven to reduce the underlying hemorrhoidal condition. In addition the prior art surgical approaches are particularly invasive, involve painful procedures and require extended recovery times. Further, the disparate range of therapies is also related to the different degree of symptoms suffered by the patient. Finally, all of these prior treatments for hemorrhoids do not address the underlying causes that gave rise to the hemorrhoidal condition initially. Since there is no attempt made for long-term prevention or the prevention of progression of symptoms once the symptoms have become established, often the condition may recur resulting in further progression and a worsening of the condition.

There is therefore a need for a standardized pharmacological approach for the treatment of hemorrhoids that serves to alleviate the symptoms, prevent progression of the symptoms and spare the patient from the need for surgery while also being convenient and easy to use.

### BRIEF SUMMARY OF THE INVENTION

In this regard, the present invention provides a novel method and system for a systematic approach to the treatment of hemorrhoids. The system of the present invention presents a novel approach to the treatment of hemorrhoids in that it provides a coherent and targeted treatment regimen for the comprehensive treatment for hemorrhoid sufferers. Further, the method and system forms a comprehensive and standardized treatment regimen that facilitates a scientific approach to the treatment of both the symptoms associated with hemorrhoids as well as the underlying causes in a conveniently packaged pharmacological solution. The system of the present invention includes the following elements:

1) Educational materials about hemorrhoids including the prevention and treatment thereof;

2) Cream for use in early stages of development for relief of minor symptoms in a manner that aids in preventing progression to more uncomfortable stages requiring surgical intervention;

3) Fiber pad wipes;

4) Cleanser; and

5) Optionally, a prescription strength ointment for relief of moderately severe symptoms of pain and swelling, the use of which can prevent progression to a stage requiring surgery.

Further, the method of the present invention is directed towards the systematic treatment of both the symptoms and the underlying causes of the hemorrhoidal condition through the use and application of the system described above.

It is, therefore, an object of the present invention to provide a method and system that serves to relieve the symptoms and discomfort caused by hemorrhoids. It is also an object of the present invention to provide a method and system that serves to relieve the symptoms as well as the underlying causes associated with hemorrhoids. It is still a further object of the present invention to provide such a system in a standardized kit that results in a unified pharmacological solution.

These together with other objects of the invention, along with various features of novelty that characterize the invention, are pointed out with particularity in the claims annexed hereto and forming a part of this disclosure. For a better understanding of the invention, its operating advantages and the specific objects attained by its uses, reference should be had to the accompanying drawings and descriptive matter in which there is illustrated a preferred embodiment of the invention.

### BRIEF DESCRIPTION OF THE DRAWINGS

In the drawings which illustrate the best mode presently contemplated for carrying out the present invention:

FIG. 1 illustrates the components of the prepackaged system of the present invention.

### DETAILED DESCRIPTION OF THE INVENTION

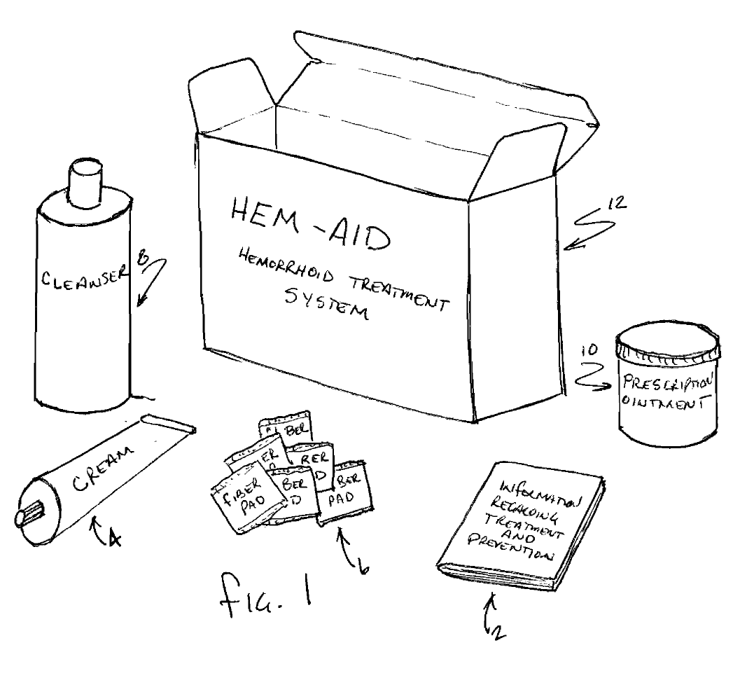

Now referring to the drawings, FIG. 1 depicts the system for a systematic approach to the treatment of hemorrhoids as disclosed in the present invention. As was stated above, there is no consensus as to a standard treatment for the prevention and treatment of hemorrhoids. As a result, the prior art approach has proven to be spotty at best causing some healthcare providers to favor surgical solutions while others resist surgery in favor of less invasive alternatives. Often two patients having an affliction that appears nearly identical may receive entirely different treatment recommendations.

In the alternative, the system of the present invention presents a novel approach to the treatment of hemorrhoids in that it provides a coherent and targeted treatment regimen for the comprehensive treatment for hemorrhoid sufferers. Further, the method and system forms a comprehensive and standardized treatment regimen that facilitates a scientific approach to the treatment of both the symptoms associated with hemorrhoids as well as the underlying causes in a conveniently packaged pharmacological solution. As was stated above the system of the present invention generally includes the following elements: educational materials 2, non-prescription cream 4, fiber pad wipes 6, cleanser 8 and an optional prescription strength ointment 10. Further, all of the above elements are prepackaged in a single box 12 for easy off the shelf dispensing and sale. In summary, the present invention is directed to a system including the above noted elements and a method of employing a comprehensive treatment regimen utilizes the above system in a manner that standardizes and simplifies the treatment of hemorrhoids.

Turning now to each of the components of the system of the present invention in detail, educational materials 2 are included in the prepackaged system in the form of a booklet, or flyer. The educational materials 2 include text and/or illustrations that provide the hemorrhoid sufferer with an overview of the underlying causes associated with their condition. Further, the educational materials 2 provide the user with guidelines for use of the comprehensive treatment system in a manner that will greatly reduce their discomfort and hasten the healing of the affected region while hopefully reducing or eliminating the instances where surgical intervention is necessary. In other words, the educational materials 2 serve both as a means for informing the user regarding their conditions as well as an instruction manual for the use of the system of the present invention in a manner that treats both the immediate symptoms as well as the underlying cause of the hemorrhoid condition.

The cream 4 for early stage treatment includes an emollient and 1% hydrocortisone cream. The combination of the emolument properties of the base vehicle and the anti-itch/anti-inflammatory properties of the hydrocortisone assist in treating early stage symptoms. The user of the system applies the cream 4 at an early stage to get immediate relief of the minor symptoms associated with early stage hemorrhoids. By applying the cream 4 in a manner that relieves the early stage itching and mild discomfort the hemorrhoids can be prevented from progressing to a more severe stage.

Fiber pads 6 are included in the system as well. The fiber pads 6 are impregnated with at least 1% hydrocortisone cream for use in application of the cream 4 as well as a preventative at the first signs of early stage symptoms. The fiber pads 6 serve as an applicator for the cream 4 as well as a stand-alone pre-medicated pad.

The fiber pads 6 are intended for use in both relieving minor symptoms as well as in personal hygiene for cleaning the affected area. In contrast, the use of standard toilet tissue for hygienic purposes is irritating and tends to aggravate the region thereby accelerating the symptoms of the sufferer. The fiber pads 6 are provided for use in place of toilet tissue and for hygienic application of the active agent throughout the day.

The cleanser 8 product includes the cleaning properties of soap but further includes a conditioner to prevent drying and restore moisture to the region. The composition of the cleanser 8 is similar in character to single formulation shampoos that contain a cleaner and conditioner in a single product. By utilizing the cleanser 8 in place of normal soap, the user can avoid the drying effect of soap that promotes symptoms of itching.

Finally, an optional prescription strength ointment 10, such as corticosteroid for example, may be used as a component of the treatment regimen. The use of the prescription ointment 10 is limited for treatment of substantial symptoms when maximum and rapid therapeutic effect is needed to prevent surgical intervention.

However, the use of this prescription ointment 10 must be limited (in terms of days) and controlled because of the high potency of the active ingredient.

In summary, the present invention is directed to a comprehensive treatment system that is prepackaged 12 in a manner that standardizes and simplifies the treatment of hemorrhoids. Further, the present invention encompasses a method of treating hemorrhoids in a standardized manner using the prepackaged 12 system of the present invention. In this manner the system and method of the present invention provide a novel and previously unrealized pharmacological product that standardizes the treatment of hemorrhoid symptoms at an early stage thereby reducing the need for surgical or otherwise invasive intervention. Further, the prepackaged nature of the system whereby all of the necessary components for effective treatment are contained therein in a simple and straightforward manner with easy to follow instructions for use enhance the potential for successful early stage treatment of the hemorrhoidal condition. For these reasons, the instant invention is believed to represent a significant advancement in the art, which has substantial commercial merit.

While there is shown and described herein certain specific structure embodying the invention, it will be manifest to those skilled in the art that various modifications and rearrangements of the parts may be made without departing from the spirit and scope of the underlying inventive concept and that the same is not limited to the particular forms herein shown and described except insofar as indicated by the scope of the appended claims.

What is claimed:

1. A prepackaged system for the treatment of chronic hemorrhoids comprising in combination:
cream for use in early stages of hemorrhoid development, said cream providing relief of minor hemorrhoid symptoms;
fiber pad wipes;
cleanser; and
educational materials, said educational materials containing information regarding the prevention and treatment of hemorrhoids and instructions regarding the use of said cream, fiber pad wipes and cleanser.

2. The prepackaged system of claim 1, further comprising:
a prescription strength ointment.

3. The prepackaged system of claim 1, said system being standardized to serve as a uniform pharmacological solution for the treatment of hemorrhoids.

4. The prepackaged system of claim 1, said system being a non-surgical treatment directed toward the remedy of both the symptoms and the underlying causes associated with hemorrhoids.

5. The prepackaged system of claim 1, wherein said system is utilized for the systematic treatment of hemorrhoids.

6. A method of treating hemorrhoids comprising:
providing a prepackaged system, said system including:
cream for use in early stages of hemorrhoid development, said cream providing relief of minor hemorrhoid symptoms;
fiber pad wipes;
cleanser; and
educational materials, said educational materials containing information regarding the prevention and treatment of hemorrhoids and instructions regarding the use of said cream, fiber pad wipes and cleanser.

7. The method of claim 6, further comprising:
employing said prepackaged system to systematically treat hemorrhoids.

8. The method of claim 6, said prepackaged system further including:
a prescription strength ointment.

9. The method of claim 6, method being standardized to serve as a uniform pharmacological solution for the treatment of hemorrhoids.

10. The method of claim 6, said method being a non-surgical treatment directed toward the remedy of both the symptoms and the underlying causes associated with hemorrhoids.
